(19) United States
(12) Patent Application Publication
(10) Pub. No.: US 2004/0146588 A1
(43) Pub. Date: Jul. 29, 2004

(54) Composition for adaptive immune system optimization and method therefor

(76) Inventor: Fanny Delia Marchosky Kogan, San Juan, PR (US)

Correspondence Address: Eugenio J. Torres, Suite 1, 1060 Ashford Ave., San Juan, PR, 00907, US

(21) Appl. No.: 10/760,969

(22) Filed: Jan. 20, 2004

Related U.S. Application Data
(60) Provisional application No. 60/441,955, filed on Jan. 22, 2003.

(51) Int. Cl. A61K 35/78

(52) U.S. Cl. 424/754; 424/766

(57) ABSTRACT

The present invention relates to a composition for the optimization of the adaptive immune system based on aged garlic extract, to a process for preparing the same, and to a method of administration or usage with a specific dosage schedule. The composition is free from unpleasant after body odors or side-effects. The invention of this disclosure uses a well-known herb/food as a supplement/medicinal agent, namely garlic, which when prepared and aged in combination with brandy alcohol, and utilized as disclosed herein, is effective with upper respiratory infections, colds, and influenza prevention for up to 2 years, easy to use, and less expensive than similar products available in the market. A method for preparing said composition is disclosed.

### RELATED APPLICATIONS

This application claims the benefit of the filing date of provisional application Ser. No. 60/441,955, filed Jan. 22, 2003, the entire disclosure of which is incorporated by reference herein as if fully set forth in its entirety.

### STATEMENT REGARDING FEDERALLY SPONSORED RESEARCH AND DEVELOPMENT

N/A

### BACKGROUND OF THE INVENTION

1. Field of the Invention

The present invention relates generally to a composition and a method of utilization of said composition to enhance and optimize the adaptive immune system's health and, more particularly, to a composition and method of utilization in a 2 month program specific dosage schedule of said composition for building up the immune defense system comprising an herb/food with multiple healing properties of colds, influenza, and upper respiratory infections.

2. Discussion of the Background

Preventive nutritional supplements are important to maintain a healthy standard of living. There is a need in today's world to reinforce our immune system in order to have good health, regardless of stressful conditions. This reinforcement of the immune system is not guaranteed by the daily intake of vitamins and minerals alone.

The composition of this invention uses the theory of low dosages in homeopathic remedies and rethinks the essence of this theory.

Homeopathy

Homeopathy is a 200-year-old healing art founded in Europe. It is a school of medicine based on the premise that “like cures like.” Practitioners believe that substances that produce certain symptoms in a healthy person can cure diseases that have similar symptoms. As an example, homeopathic physicians might use a natural product known to cause stomach irritation in a healthy person and administer it in a greatly diluted form (from three to 100,000 times) to treat a stomach ulcer. Homeopathy is gaining renewed popularity and recognition in the United States. Complementing rather than opposing traditional medicine, homeopathy offers a therapeutic alternative that is natural, safe, and effective. Low dose homeopathic ingredients help natural bodily mechanisms correct underlying dysfunction and relieve disease symptoms.

Homeopathy dates back to the work of Samuel Hahnemann, a German doctor seeking a gentler alternative to the often-brutal treatments of the day. Hahnemann argued that any substance that produces a symptom in a healthy person could cure that symptom in a sick person. Today, the homeopathic medicine cabinet contains products derived from sulfur, snake venom, duck liver, squid ink, mercury, arsenic, belladonna, crude oil, and a host of other natural compounds—many of them identified by Hahnemann.

Hahnemann also believed that even minute doses could cure, and he diluted his preparations multiple times before giving them to patients. Modern homeopathic mixtures can be diluted a million fold or more—often so many times that no active ingredients are left.

With the foregoing in mind, yet not to cure a disease but instead, to prevent, shorten the duration and/or lessen the severity of many illnesses, using the principle of small doses of a renowned herb/food with multiple healing properties in a 2 month program specific dosage schedule to build the immune defense, the composition of this invention results in a build up of the adaptive part of our immune defense system. Thus, the present invention is not a homeopathic remedy—since it does not heal any diagnosed disease, but prevents the onset for up to 2 years.

Immune System

The adaptive part of the immune system is extremely complex and only partly understood. Its function is to produce specific defenses against a range of different invading organisms or tumor cells. Broadly, however, it first must recognize part of the invading organism or tumor cell as an antigen (a protein that is foreign or different from any natural body protein) . A response (either humoral or cellular) is then mounted against the antigen. The humoral response consists of the production of soluble proteins, called antibodies or immunoglobulins, manufactured by cells called B-lymphocytes. Cellular responses center around the activities of the cells called T-lymphocytes.

Every day of our lives, our bodies are bombarded with multiple assaults from many different sources; in fact, in the last century alone, our exposure to toxins has gone up thousands of times. Only an incredibly strong immune system—the body's final line of defense against all forms of illness—can successfully handle the by-products of each one of these toxic substances. By combining a variety of factors and supporting the immune system from various angles, one can safely stimulate the immune system, while feeding and nourishing it at the same time. Through this multifaceted approach, not only can the immune response be increased, but also it is ensured that the system remains balanced, healthy, and ready for the next job that comes along.

As the child grows, he or she encounters organisms that overcome the innate defenses and thus cause disease. Then, the second line of immune defense, called the adaptive immune system, comes into play. As the name implies, this system adapts its response specifically to fight the invading microorganism. In addition, it retains a memory of the invader so those defenses can be rallied instantly in the future. The person is then said to have acquired immunity to the infection. If these microorganisms invade again, they are quickly recognized and dealt with (which explains why diseases such as measles and diphtheria rarely affect the same person twice).

When the immune defenses aren't prepared, unwanted viruses work around the system failures, microbes make themselves at home, and toxins take hold. The immune system's job is to recognize and maintain balance in every part of the body. The really amazing thing is, if it has the right nourishment that is exactly what it does.

Plant Medicine

All biological systems are complex, and human health and disease are no exception. Using single active compounds to attack disease limits our options. Medicine has made important advances against acute infectious diseases because of these active compounds, such as penicillin. However, chronic diseases, such as heart disease and cancer, often involve many body systems and processes simultaneously. This is one of the main reasons why single active compounds are not always effective in these cases. A single medicinal plant often contains hundreds of compounds. These synergistic natural remedies are ideal for promoting the body's regulatory mechanisms and restoring health. They meet complexity with complexity and address major processes of disease—such as oxidation, inflammation, and malnutrition—at their source. Aged garlic extract is a logical choice for protecting against the dominant health concerns today. No man-made medicine available today has the range of therapeutic benefits of aged garlic extract.

Garlic has been used effectively through the centuries for a variety of concerns and is probably one of the best known herbs/foods. Garlic is one of the strongest medicinal plants on earth. It is a supreme infection fighter capable of killing all bacteria, virus and fungus on contact. The medicinal use of Garlic has been documented since the beginning of recorded history. From the Egyptians and Greeks to the Romans, Garlic was used as both food and strong medicine to strengthen and heal the body. In research, it has been diluted 1 part in 125,000 and still killed bacteria. In fact, just the odor has proven to be highly antibacterial. It is an extremely effective and powerful broad-spectrum antibiotic, which means, it kills all types of bacteria on contact, gram positive and gram negative. Garlic's use as an antibacterial drug in Russia is so esteemed it has been nicknamed Russian penicillin. Garlic has been proven to destroy many types of bacteria including Streptococcus, Staphylococcus, Thypoid, Diphtheria, Cholera, E. Coli, Bacterial Dysentery (travelers' diarrhea), Tuberculosis, Tetanus, Rheumatic bacteria and many others. Garlic is also a very powerful antiviral agent proven in the laboratory, which really makes it a specific for colds, influenza, and upper respiratory infections. Many doctors feel it is the cure for the common cold. It destroys various viruses that cause upper respiratory infections and influenza, the ones that antibiotics are useless against. Garlic destroys on contact the viral infections of Measles, Mumps, Mononucleosis, Chicken Pox, Herpes Simplex # 1 and # 2, Herpes zoster, Viral Hepatitis, Scarlet fever, Rabies and others.

Unlike chemical antibiotics that kill off the millions of friendly bacteria that our body needs, Garlic only kills the bad guys and even promotes and increases our healthy bacteria. Garlic is also a powerful antifungal and literally destroys any antigen, pathogen and any harmful or disease causing microorganism that can hurt you.

Garlic has demonstrated to be a natural antibiotic, a great blood purifier, a remedy against hypertension, an anti-inflammatory, a great auxiliary to lower cholesterol, stops fermentation, is good against cardiovascular problems, it has anti-rheumatic factors, and it has more than two hundred factors that humans can use to combat their maladies. Garlic is one of the most valuable healing foods in nature's garden, and is highly recommended for a long and healthy life.

As discussed above, over the thousands of years of garlic's use, a remarkable wide range of medical benefits have been recorded. This has led many medical experts—who have been trained to target specific drugs for specific pathologies—to dismiss garlic. As the consistent influx of reports from all over the world continue to challenge their beliefs, they have looked to their own research procedures to confirm or deny these actions. As a result, the most sophisticated and modern scientific studies have demonstrated the value of garlic, specifically aged garlic extract, for the prevention and treatment of cardiovascular disease and cancer, aged garlic extract has also been found to benefit a number of other conditions. The remarkably diverse actions of aged garlic extract include immune system enhancement; allergy relief, relief of stress and fatigue; improvement of learning, behavior, and memory; reduced brain damage during stroke; protection of the liver; promotion of intestinal health; and heavy metal removal.

Like all systems of the human body, the immune system is complex and interactive. The first line of defense against the outside environment includes the skin, the mucus membranes of the nose, mouth, and lungs, and the lining of the digestive tract. As has been shown in cancer studies, aged garlic extract protects the sensitive membranes of the intestines from chemical therapy-induced damage. This is one of the strongest challenges that our intestines may face. The second line of defense involves the macrophages, which circulate through the body and engulf foreign matter and wastes that would otherwise threaten the inner environment. These cells are vital to our health. In infectious diseases and inflammation, macrophages are among the first cells on the scene to protect us; aged garlic extract makes macrophages more mobile and active. Aged garlic extract also supports the humoral immune system, in which T-cells identify invaders, natural killer cells attack them, and B-cells help create antibodies to resist them. In a clinical trial, aged garlic extract was 140 percent more effective than fresh garlic, and 155 percent more effective than garlic powder capsules, at enhancing natural killer cell activity. In a study on people with AIDS, aged garlic extract was shown to raise natural killer cells to their normal, healthy levels in all subjects after only six weeks. It was also noted that these people found relief from diarrhea, candidiasis, and respiratory inflammation with recurrent fever. In addition, their recurrent cycles of genital herpes were interrupted. The last example confirms the antiviral benefits of aged garlic extract. The antiviral actions of aged garlic extract have also been tested against influenza. Not only does aged garlic extract significantly reduce the intensity of the flu, but also it was found to be as effective as a vaccine. The immune-enhancing actions of aged garlic extract have been formally tested against those of other garlic preparations, and have consistently outperformed them.

Therefore, it can be appreciated that there exists a continuing need for a new and improved aged garlic extract based composition for the optimization of the adaptive immune system. In this regard, the present invention substantially fulfills this need. The present invention overcomes the inability of prior art to provide an aged garlic extract based composition for the optimization of the adaptive immune system.

Various novel garlic based compositions have recently been disclosed, for instance, U.S. Pat. No. 6,146,638 to Kakimoto, et al. discloses a fermented garlic composition, yet none provides a faster, cost efficient, healing with no side effects as the composition of the present invention. None of the art considered above, taken either simply or in combination teach the use of aged garlic extract for the optimization of the adaptive immune system.

### SUMMARY OF THE INVENTION

The object of the present invention is to provide a new composition and a method for its use in a 2 month program specific dosage schedule for optimizing the adaptive immune system.

A further object of the present invention is to provide a composition that is completely natural and safe and free of side effects.

Another object of the present invention is to assist people in maintaining optimal health for a year or longer after completing a two month program.

Still another object of the present invention is to provide a composition and method therefor that programs the continuous and rapid stimulation of the body's natural capacity to heal itself, self balance, shortens the duration and/or lessens the severity of many illnesses, including colds, coughs, flu, and influenza.

A further object of the present invention is to provide a composition and method therefor that eliminates viruses, bacteria, colds, flu, and influenza.

Another object of the present invention is to provide a composition and method therefor that builds, fortifies, and activates the adaptive immune system and is also easy to prepare.

A further object of the present invention is to provide a composition and method therefor wherein results render the person no longer fertile ground for bacteria and viruses to grow and optimal health sustained homeostasis, prevention-protection for an extended period, is achieved after the prescribed completed program of two months without repetition required.

Still a further object of the present invention is to provide a unique composition and method therefor that is natural, safe, side-effect free and successfully meets and solves for a lengthy time, health challenges from the constantly changing, threatening, toxic environment of today's world.

Another object of the present invention is to provide a composition that is of low cost.

The composition and method themselves, both as to their construction and mode of operation, will be best understood, and additional objects and advantages thereof will become apparent from the following description of the preferred embodiments.

When the word “invention” is used in this specification, the word “invention” includes “inventions”, that is, the plural of “invention”. By stating “invention”, the Applicant does not in any way admit that the present application does not include more than one patentable and non-obviously distinct invention and Applicant maintains that the present application may include more than one patentable and non-obviously distinct invention. The Applicant hereby asserts that the disclosure of the present application may include more than one invention, and, in the event that there is more than one invention, that these inventions may be patentable and non-obvious one with respect to the other.

Further, the purpose of the accompanying abstract is to enable the U.S. Patent and Trademark Office and the public generally, and especially the scientists, engineers, and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

### DESCRIPTION OF THE PREFERRED EMBODIMENT

The present invention encompasses a new composition and method of usage with a specific variable dosage schedule for the optimization of the adaptive immune system. Individual results may last from six months to two years.

An aged, nutraceutical supplement elixir is obtained following the process of preparation. In the preferred embodiment, the composition according to the present invention comprises from about 0.01 to about 40 weight percent of an herb-food-garlic (Allium sativum L., such as, for example, that which belongs to the Liliaceae Allium family) wherein said herb-food garlic is crushed to produce juice, and from about 30 to about 80 weight percent of clear brandy alcohol, wherein said herb-food garlic and said clear brandy alcohol are both aged in a temperature of 33 degrees F. to about 40 degrees F. for from about 200 to about 300 hours.

The second step requires straining the garlic and removing the herb from the composition, thus leaving only the brandy alcohol and the garlic extract and aging at a temperature of 33 degrees F to about 40 degrees F. for from about 40 to about 60 hours.

In an alternative embodiment, the composition may comprise additives such as a taste improver, flavoring agent or a buffer.

The product obtained is an aged garlic composition extract, ready for consumption, in a prescribed oral formulation specific variable drop dosage program to be followed three times a day with meals, for two months, regardless of age or body weight, and with no resulting after body odor. Results from this product and method is an optimal adaptive immune system, also known as the second line of the immune defense lasting from 6 months to 2 years. The drop dosages vary from 1 to 25 drops. The drops may be added to any carrier such as water, milk, or juice.

Upon completion of the supplement product program, the body is “fine tuned” and easily recognizes any new threat to its homeostasis, quickly adapting and optimizing all defenses. Likened to “bodyguards” circling and protecting from every possible invader enemy entrance, cutting short symptoms or effects of disease—any intent of attack gets knocked out in hours instead of weeks. A health-utopia state is therefore viable.

All of the patents, patent applications, and publications recited herein, and in the Declaration attached hereto, if any, are hereby incorporated by reference as if set forth in their entirety herein. The components disclosed in the various patents, patent applications, and publications, disclosed or incorporated by reference herein may be used in the embodiments of the present invention, as well as equivalents thereof.

All, or substantially all, of the components and methods of the various embodiments may be used with at least one embodiment or all of the embodiments, if more than one embodiment is described herein.

The details in the patents, patent applications, and publications may be considered to be incorporable at applicant's option, into the claims during prosecution as further limitations in the claims to patentably distinguish any amended claims from any applied prior art.

Thus, there has been shown and described a composition for adaptive immune system optimizer and method of usage with specific variable dosage which fulfills all the objects and advantages sought therefor. Although only a few exemplary embodiments of this invention have been described in detail above, those skilled in the art will readily appreciate that many modifications are possible in the exemplary embodiments without materially departing from the novel teachings and advantages of this invention. For instance, a different carrier may be used or the composition of the present invention may also find application for treatment of certain other diseases. Accordingly, all such modifications are intended to be included within the scope of this invention as defined in the appended claims and their legal equivalents. In the claims, means-plus-function clauses, if any, are intended to cover the structures described herein as performing the recited function and not only structural equivalents but also equivalent structures.

Nothing in this application is intended to limit additional compositions that may be added to or used in conjunction with composition of present invention. Although therapeutics of present invention may be orally ingested, any suitable solution, any other suitable agent, carrier or delivery vehicle may be used. A wide variety of compositions including antibiotics, antibiotic creams, or non-toxic pharmaceutical carriers or vehicles, or the like, and in any suitable form such as a liquid, solid, semi-solid, ointment, lotion, paste, or the like may also be used where advantageous.

Nothing in the application is intended to limit the devices and methods used to facilitate application of composition of present invention. Devices and methods currently existing or to be developed in the future could be employed.

What is claimed is:

1. A composition for optimization of the adaptive immune system, comprising:
(a) from about 0.01 to about 40 weight percent garlic; and
(b) from about 30 to about 80 weight percent clear brandy alcohol.

2. The composition of claim 1, comprising, preferably 20 weight percent garlic.

3. A method for preparing the composition of claim 1, comprising the steps of:
(a) Placing the garlic inside a container filled with clear brandy alcohol;
(b) Letting the garlic age inside said container at a temperature of from about 33 to about 40 degrees F. for from about 200 to about 300 hours, thereby allowing the garlic juice to mix with the brandy alcohol;
(c) Removing the garlic from the container after allowing the garlic juice to drain from inside the garlic;
(d) Aging the resulting composition at a temperature of from about 33 to about 40 degrees F. for approximately from about 40 to about 60 hours.

4. A method for administering the composition made by the process of claim 3 for optimization of the adaptive immune system, comprising the steps of:
(a) Selecting the proper dosification from the instructions of detailed dosifications tables, wherein said dosifications consist of from about 1 to about 25 drops of the composition varying according to a specific dosage schedule;
(b) Administering proper dosage for a period of 2 months three times a day with a meal, regardless of age or body weight.

5. The method of claim 4, further comprising the step of adding the proper dosage to a carrier.

6. The method of claim 5, wherein said carrier is selected from a group consisting of water, milk, and juice.
